(19) United States
(12) Patent Application Publication
(10) Pub. No.: US 2026/0027287 A1
(43) Pub. Date: Jan. 29, 2026

(54) SOAKING OPERATIVE SYSTEM, DEVICE, AND METHODS

(71) Applicant: Sterling Innovations, LLC, Scottsdale, AZ (US)

(72) Inventor: Danton S. Dungy, Scottsdale, AZ (US)

(21) Appl. No.: 19/284,057

(22) Filed: Jul. 29, 2025

Related U.S. Application Data
(60) Provisional application No. 63/676,859, filed on Jul. 29, 2024.

(51) Int. Cl. A61M 5/14; A61M 5/158; A61M 19/00; A61M 31/00

(52) U.S. Cl. CPC A61M 5/1407; A61M 5/158; A61M 19/00; A61M 31/002; A61M 2205/02

(57) ABSTRACT

A system, device, and method for dispersing medicines, fluids, or liquids to a patient are disclosed. A device with a series of porous tubes that dispense a liquid directly to a body cavity, wound, or within a patient's body is disclosed. Further, a method for dispensing medicines, fluids, or liquids to a patient using such a device is disclosed.

### CROSS-REFERENCE TO RELATED APPLICATION

This application claims priority to U.S. Provisional Patent Application No. 63/676,859, filed Jul. 29, 2024, and entitled “SOAKING OPERATIVE SYSTEM, DEVICE, AND METHODS,” the disclosures of which are hereby incorporated by reference herein.

### TECHNICAL FIELD

This disclosure relates to a system, device, and method of delivering medicine, fluids, or liquids, and in particular a device for administering medicine, fluid or liquid to explicit locations, such as body cavities or post-surgery operative fields.

### BACKGROUND

In the health, medical, pharmaceutical and/or life science industry, there often is a need to administer medicine, fluids, or liquids to patients. In particular, it is common post operation for patients to take various medicines to reduce pain and ensure proper healing. Typically, medicine is taken orally or intravenously using IVs and other injecting means. Further, there is often a need to administer saline or other fluids to patients following surgery. Similarly, fluids and saline are often administered using IVs which direct the fluids to the blood stream. Current methods fail to efficiently apply these fluids or liquids directly to the location of the wound, body cavity or post surgery operative field. While topical applications and administration of fluids or medicine by a needle are often used, these methods are less effective and difficult for patients to administer without the help of a medical professional.

Accordingly, there is a need for a system, apparatus and/or method to directly administer medicine, fluids and liquids to specific locations, such as a body cavity or post surgery operative field.

### SUMMARY

The present invention is directed at a device that can be implanted into a patient's body to dispense liquid containing medicines, fluids, or other desirables compositions to specific locations within a patient's body. In one embodiment, the device is a soaking operative device for applying a liquid substance comprising a hollow member with two or more tubes that are in contact with a contact point. The hollow tubes comprise one or more applicator slots and inlets that are positioned on one or more of the tubes and an inlet attachable to a container holding the liquid substance to be dispensed by the system.

In various embodiments, the hollow member may comprise one or more of the tubes in a first direction and one or more of the tubes in a second direction. The tubes in the first direction may be connected with the tubes in the second direction at the tube contact point. The hollow member may be inserted below a patient's skin and the inlet is inserted through the patient skin by an application needle. The inlet may be connected to a compression connector, the compression connector configured to connect to a syringe, the syringe for providing the liquid substance to the hollow member. The two or more tubes of the hollow member may be made of an absorbable suture material. The two or more tubes may comprise a first end and a second end, wherein the first end and the second end of the two or more tubes are sealed. The liquid substance may comprise at least one or more of: an anti-inflammatory; an anesthetic; an analgesic; a saline solution; a chemotherapeutic medication; and an antibiotic solution.

In another embodiment, a soaking operative system for applying a liquid substance is disclosed. The system may comprise a hollow member comprising two or more tubes. The one or more of the tubes may be in a first direction and one or more of the tubes is in a second direction, wherein the one or more tube in the first direction is connected with the one or more tube in the second direction at a tube contact point. The system may further comprise an inlet positioned on one of the tubes, the inlet attachable to a container including the liquid substance.

In another embodiment, a method of applying a liquid substance with a soaking operative system is disclosed. That method comprises the steps of inserting a hollow member under the patient's skin, providing a liquid substance to an inlet attached to the hollow member and dispensing by liquid substance though one or more applicator slots on the hollow member.

### BRIEF DESCRIPTION OF THE DRAWINGS

Other systems, methods, features, and advantages of the present invention will be apparent to one skilled in the art upon examination of the following figures and detailed description. Component parts shown in the drawings are not necessarily to scale and may be exaggerated to better illustrate the important features of the present invention.

FIG. 1 illustrates a soaking operative system comprising an application needle, in accordance with various embodiments;

FIG. 2 illustrates a soaking operative system comprising a compression connector, in accordance with various embodiments;

FIGS. 3A and 3B illustrate a compression connector, in accordance with various embodiments;

FIG. 4 illustrates a tube contact point of a soaking operative system, in accordance with various embodiments;

FIG. 5 illustrates an exemplary soaking operative system, in accordance with various embodiments; and

FIG. 6 illustrates a flowchart for a method of applying a liquid substance with a soaking operative system, in accordance with various embodiments.

### DETAILED DESCRIPTION

For simplicity and clarity of illustration, the drawing figures illustrate the general manner of construction, and descriptions and details of some features and techniques may be omitted. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denotes the same elements.

Disclosed herein are systems, devices and/or methods for applying or soaking a area below the skin, such as a body cavity or post surgery operative field, with a liquid substance. The soaking operative system comprises multiple tubes which are at least partially connected. The soaking operative system comprises an inlet configured to receive the liquid substance and disperse the liquid substance through the tube system and out of applicator holes. The applicator holes applying the liquid to an area under a patient's skin. In various embodiments, the liquid substance may include, but not be limited to an anesthetic or analgesic, such as an anti-inflammatory, local anesthetic, narcotic, or non-narcotic medication; a saline solution; an antibiotic solution; or a chemotherapeutic medication.

The soaking operative system delivers a liquid substance directly to the targeted area below a patient's skin. In various embodiments, the hollow member may be placed below a patient's skin within an operative bed of tissue. The application by the soaking operative system disperses the liquid substance below the skin, allowing for more direct and rapid application and relief. For example, by applying an analgesic, such as an NSAID or local anesthetic, using the soaking operative system, the analgesic will apply directly to the body cavity and offer more rapid and accurate relief. Further, the soaking operative system may reduce the amount of a liquid substance applied systemically to a patient when compared to other methods, such as intravenous or oral administration.

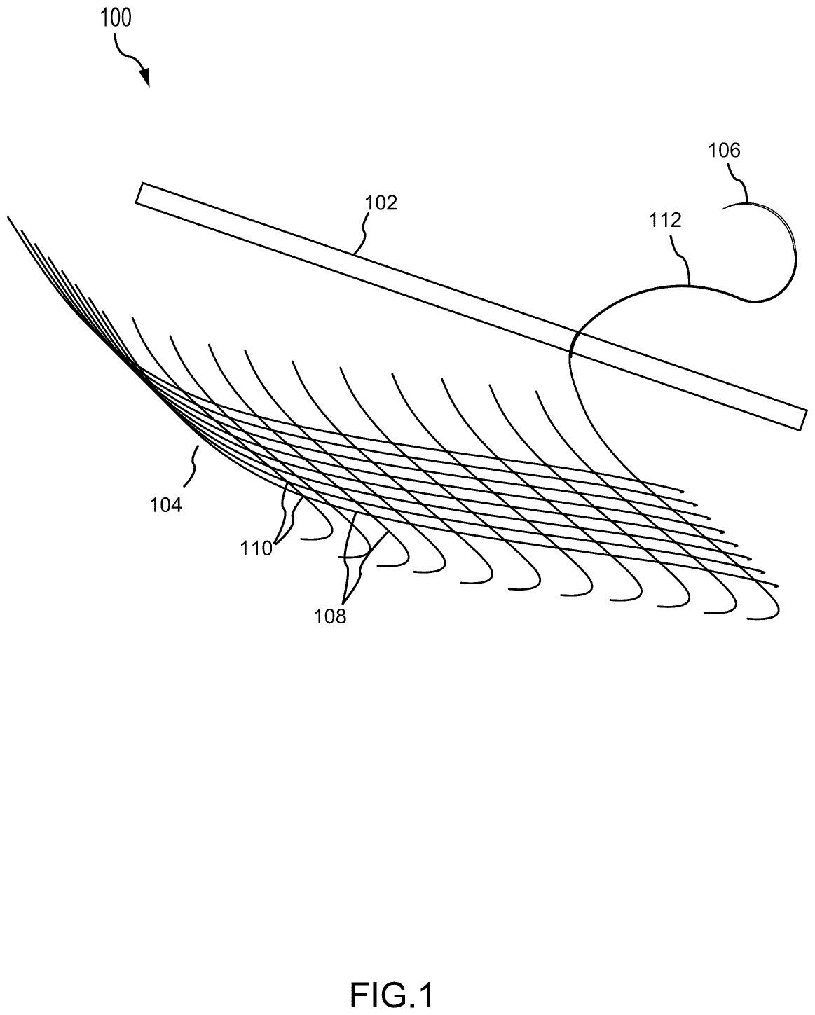

With respect to FIG. 1, a soaking operative system 100 is shown, in accordance with various embodiments. In various embodiments, the soaking operative system 100 may comprise a hollow member 104. In various embodiments, the hollow member 104 may comprise one or more tubes 108. In various embodiments, the tubes 108 may connect at tube contact points 110.

In various embodiments, the tubes 108 of the hollow member 104 may be arranged similar to a netting or mesh-like structure. In various embodiments, the tubes 108 may be hollow or tubular. For example, the tubes 108 may be configured to receive a liquid substance and disperse the liquid substance through the hollow member 104. In various embodiments, the tubes 108 may be made of a dissolvable material, such as an absorbable suture material. For example, the tubes 108 may be made of a material that dissolves, such as polydioxanone or poliglecaprone, when inserted below the patient's skin 102. The hollow member 104 may be placed under the skin 102 in a body cavity such as in a wound, and/or post surgery operative field. In various embodiments, the tubes 108 may dissolve after a reasonable period of time to allow use of the soaking operative system 100 when inserted below the skin 102, such as 20 days to 180 days. In various embodiments, the tubes 108 may alternatively be made of a non-absorbable material that may be removed after use.

In various embodiments, the tubes 108 may be various sizes or diameters. For example, the tubes 108 may be approximately 1 mm, 1.5 mm and/or 2 mm in outer diameter. It can be appreciated that the tube 108 may be larger or smaller in diameter for various applications. In various embodiments, the tubes 108 may be between 0.5 mm and 3 mm in outer diameter.

In various embodiments, tubes 108 may be connected wherein the hollow member 104 comprises one or more of the tubes 108 in a first direction and one or more of the tubes 108 in a second direction. In various embodiments, tubes 108 in the first direction may be connected with tubes 108 in the second direction at the tube contact point 110. In various embodiments, the tubes 108 in the first direction may be perpendicular to the tubes 108 in the second direction. Further, in various embodiments, two or more of the tubes 108 may be parallel to each other. In various embodiments, the tube contact points 110 may be positioned such that they are evenly spaced to allow even distribution. In various embodiments, the tubes 108 may connect in a spiral like configuration.

In various embodiments, the hollow member 104 may come in various sizes for use in various size or areas of a body cavity or post surgery operative field. In various embodiments, the hollow member 104 may be large 30×30 cm, medium 20×20 cm, or small 10×10 or similar sizes. In various embodiments, the hollow member 104 may be shaped to fit within a surgical knee wound, abdominal cavity or other body part. In various embodiments, the hollow member 104 may be cut to a certain size.

The hollow member 104 may comprise any suitable number of tubes 108. The hollow member 104 may comprise tubes 108 spaced closer together or further apart for various applications. In various embodiments, tubes 108 may be spaced such that the tubes 108 in a first direction and/or second direction are approximately 5 mm apart. In various embodiments, the tubes 108 may be spaced such that the tubes 108 in a first direction and/or second direction are approximately 10 mm apart. In various embodiments, the tubes 108 may be spaced such that the tubes 108 in a first direction and/or second direction are between 3 mm and 15 mm apart.

In various embodiments, the diameter and spacing of the tubes 108 may depend on the application desired. For example, the size and spacing of the tubes 108 may depend on the size of the surgical cavity (e.g. the diameter of the tubes 108 may be smaller size in diameter and/or closer spacing for a breast biopsy cavity than a hip replacement cavity). Further, in various embodiments, diameter and spacing of the tubes 108 may depend on the specific medication being delivered, also referred to herein as liquid substance. It can be appreciated that the diameter and spacing of the tubes 108 may adjust the flow rate and volume of the liquid substance, therefore a particular diameter and spacing of the tubes 108 may be used based on desired flow rate. For example, the diameter and spacing of the tubes 108 for using a local anesthetic may be different than once daily antibiotics.

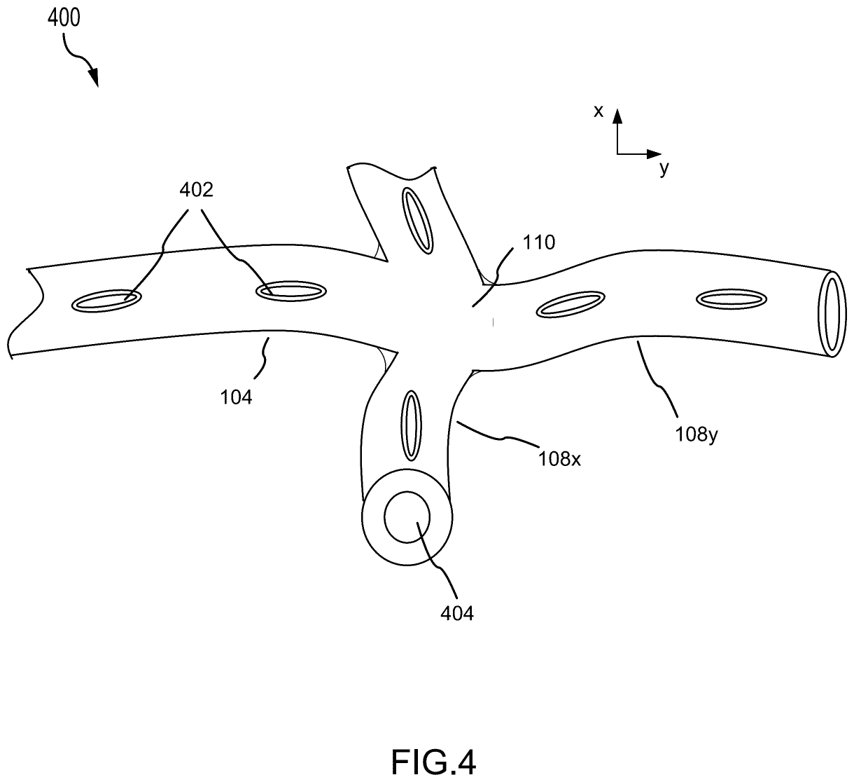

In various embodiments, the hollow member 104 may be cut to a size and the ends of the tubes 108 may be sealed. In various embodiments, the ends of the tubes 108 may be cut and sealed by a safe sterile heating element. In various embodiments, each of the tubes 108 may comprise a first end and a second end, wherein the first end and the second end of the two or more tubes 108 is sealed, and the first end of one of the tubes 108 is connected to the inlet 112. In various embodiments, the hollow member 104 is configured to disperse the liquid substance through the one or more applicator slots 402, as described with reference to FIG. 4.

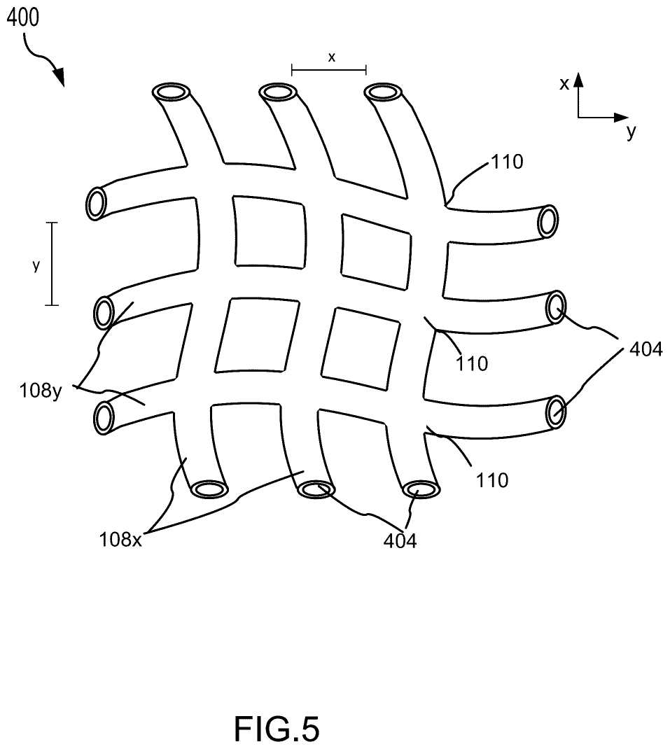

In various embodiments, the tubes 108 may be cut and not sealed. For example, as described in more detail with reference to FIG. 5, the tubes 108 may comprise hollow openings 404, wherein the liquid substance is dispersed through the hollow chamber 404.

In various embodiments, the soaking operative system 100 may be inserted below a patient skin 102. For example, the soaking operative system 100 may be within a wound, body cavity or other area below the patient's skin 102. Typically, the body cavity will be closed over the soaking operative system 100, which will allow the liquid substance being administered to stay within the patient to have a direct effect on the local tissue.

In various embodiments, the hollow member 104 may comprise an inlet 112. In various embodiments, the inlet 112 may be positioned on one of the tubes 108. In various embodiments, the inlet 112 may configured to receive the liquid substance. In various embodiments, the inlet 112 is inserted through the patient skin 102 by an application needle 106. For example, the application needle 106 is used to pass the inlet 112 to the external side of the skin 102. In various embodiments, the application needle 106 may be inserted exiting through the skin at least 3 cm from the incision wherein the hollow member 104 was inserted. The application needle 106 may be removed after insertion through the skin 102. In various embodiments, the application needle 106 may be attached to inlet 112 via a taper fit and/or a medical-grade adhesive.

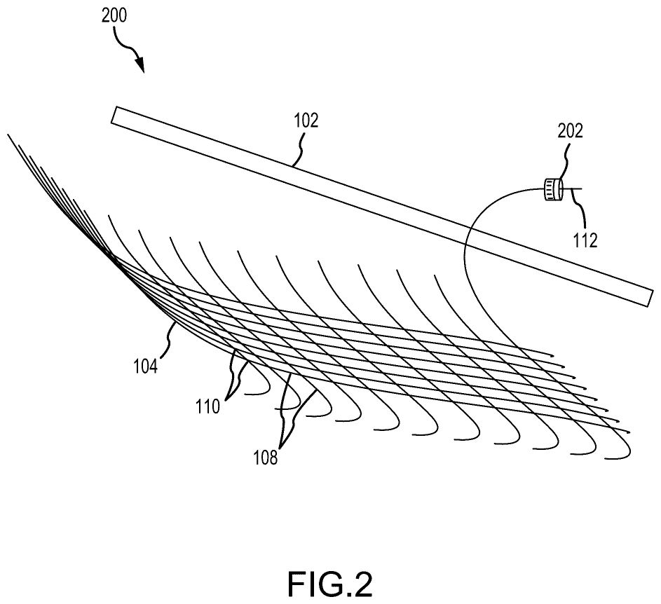

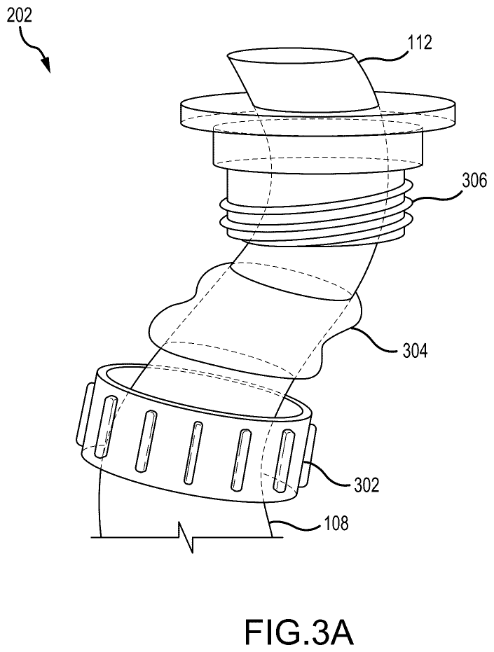

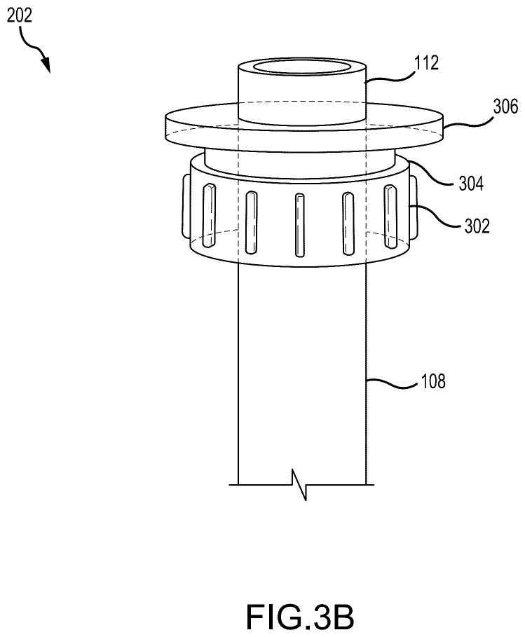

With reference to FIG. 2, a soaking operative system 200 comprising a compression connector 202 is shown. The soaking operative system 200 may be similar in design and function as soaking operative system 100. In various embodiments, the inlet 112 may be attached to the compression connector 202. The compression connector 202 may be any suitable device for connecting to the liquid substance. A compression connector 202, in accordance with various embodiments, is described in greater detail with reference to FIGS. 3A and 3B. In various embodiments, after the hollow member 104 may be inserted below the skin 102 and the application needle 106 is removed, the compression connector 202 is attached to the tube 108 in proximity to the to the inlet 112. In various embodiments, the compression connector 202 is configured to compress or seal closed the tube 108 which is connected to the inlet 112.

With reference to FIGS. 3A and 3B, compression connector 202 is shown, in accordance with various embodiments. In various embodiments, the compression connector 202 may connect tube 108 leading to the inlet 112. The compression connector 202 may comprise a nut 302, a ring 304, and an attachment 306. In various embodiments, the ring 304 may be made of a compressible material. In various embodiments, the ring 304 may be made of silicone or other compressible plastic. In various embodiments, the nut 302 may connect with the attachment 306. For example, the attachment 306 may comprise threads configured to screw into the nut 302, wherein the ring 304 is configured to compress between the nut 302 and attachment 306. In various embodiments, the nut 302 and attachment 306 may be connected to tighten onto the tube 108. In various embodiments, the compression connector 202 may tighten onto the tube 108 to compress or seal the tube 108. In various embodiments, the compression connector 202 may be configured to connect to a liquid delivery device such as a luer lock syringe.

In various embodiments, the compression connector 202 may be attached or removed from the inlet 112 in a “no compression state” For example, when the compression connector 202 is attached to the inlet 112, the nut 306 may not be tightened to the attachment 306. For example, the compression connector 202 may be in a “no compression state” allowing for the compression connector 202 to easily be attached or removed from the tube 108 connected to the inlet 112. In various embodiments, the compression connector may be in a slight compression or “open state,” which may allow for the compression connector 202 to adhere to the tube 108, wherein the compression connector 202 allows the tube 108 to still be open for liquid flow. For example, the attachment 306, comprising a screw mechanism may adhere to the tube 108, while not pinching the tube 108 when the compression connector 202 is in a “open state.” In various embodiments, and as discussed, the compression connector 202 may be in fully compressed or “closed state” which may seal the tube 108. For example, the compression connector 202 may be in a “closed state” when the nut 302 and attachment 306 are tightened together such that the tube 108 is sealed.

With reference to FIG. 4, a portion of a soaking operative system 400 in accordance with various embodiments is shown. In various embodiments, the tubes 108 may comprise applicator slots 402. In various embodiments, the applicator slots 402 may be configured to release the liquid substance. In various embodiments, the applicator slots 402 may be smaller than a millimeter. In various embodiments, the applicator slots 402 may be microscopic in size or smaller than.1 millimeters. In various embodiments, the liquid substance may enter the hollow member 104 through the inlet 112 and exit through the applicator slots 402. The applicator slots 402 may be positioned on various sides of tubes 108. In various embodiments, the applicator slots 402 may be spaced to evenly apply and disperse the liquid substance. In various embodiments, tubes 108 may comprise a hollow chamber 404. In various embodiments, the hollow chamber 404 may support the flow of the liquid substance. In various embodiments, the tube contact points 110 may be hollow and internally connect tubes 108 in a first direction with tubes 108 in a second direction. For example, the hollow chamber 404 of a tube 108x in a x direction may be internally connected with the hollow chamber 404 of a tube 108y in a y direction.

With reference to FIG. 5, a soaking operative system 400 comprising hollow ends 404 is shown in accordance with various embodiments. In various embodiments, the tubes 108 may comprise hollow chambers 404, wherein the hollow chambers 404 are configured to release and disperse the liquid substance. For example, in various embodiments, the hollow member 104 may comprise tubes 108 wherein the ends of the tubes 108 are open and not sealed, wherein the liquid substance is released by the ends of the tubes 108.

In various embodiments and as shown in FIG. 5, the tubes 108y are in a y direction, may be spaced by a spacing “y” and the tubes 108x in an x direction may be spaced by a spacing “x.” As discussed in reference to FIG. 1, the spacing between each of the tubes 108x or 108y may be between 3 mm and 15 mm apart. In various embodiments, the spacing x may be approximately the same distance as the spacing y. In various embodiments, the spacing x may be smaller or larger than spacing y. In various embodiments, the spacing x and spacing y may be approximately 5 mm. In various embodiments, the spacing x and spacing y may be approximately 10 mm.

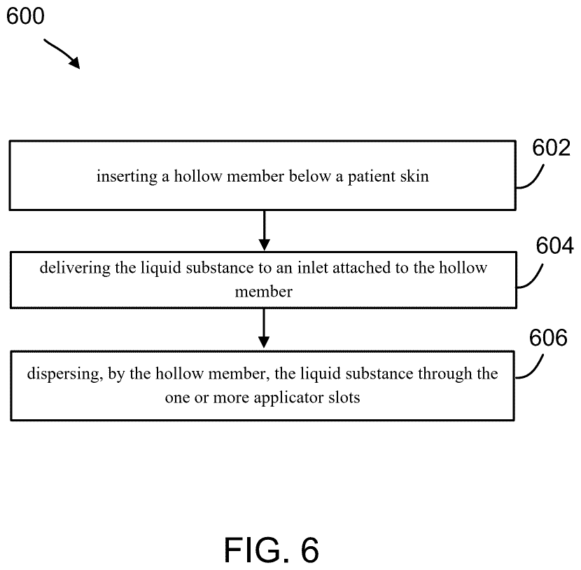

With reference now to FIG. 6, a method of applying a liquid substance with a soaking operative system 600 is shown. The method 600 comprises inserting a hollow member below a patient's skin (step 602). The hollow member may comprise two or more tubes, the two or more tubes comprising one or more applicator slots. The method 600 may comprise deliver the liquid substance to an inlet attached to the hollow member (step 604). The method 600 may comprise dispersing, by the hollow member, the liquid substance through the one or more applicator slots (Step 606).

Exemplary embodiments of the methods/systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

The terms “couple,” “coupled,” “couples,” “coupling,” and the like should be broadly understood and refer to connecting two or more elements mechanically and/or otherwise. Two or more electrical elements may be electrically coupled together, but not be mechanically or otherwise coupled together. Coupling may be for any length of time, e.g., permanent or semi-permanent or only for an instant. The absence of the word “removably,” “removable,” and the like near the word “coupled,” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching may be used throughout the figures to denote different parts but not necessarily to denote the same or different materials.

Methods, systems, and articles are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A soaking operative device for applying a liquid substance comprising:
a hollow member, wherein the hollow member comprises two or more tubes, wherein the two or more tubes are in contact at a tube contact point;
one or more applicator slots on two or more of the tubes; and
an inlet positioned on one of the tubes, the inlet attachable to a container including the liquid substance.

2. The device of claim 1, wherein the hollow member comprises one or more of the tubes in a first direction and one or more of the tubes in a second direction, wherein the tubes in the first direction are connected with the tubes in the second direction at the tube contact point.

3. The device of claim 1, wherein the hollow member is connected to an application needle, wherein the hollow member is configured to be inserted below a patient's skin and the inlet is inserted through the patient skin by an application needle.

4. The device of claim 1, wherein the inlet is connected to a compression connector, the compression connector configured to connect to a syringe, the syringe for providing the liquid substance to the hollow member.

5. The device of claim 1, wherein the two or more tubes of the hollow member are made of an absorbable suture material.

6. The device of claim 1, wherein the two or more tubes comprise a first end and a second end, wherein the first end and the second end of the two or more tubes is sealed.

7. The device of claim 1, wherein the liquid substance comprises at least one or more of:
an anti-inflammatory;
an anesthetic;
an analgesic;
a saline solution;
a chemotherapeutic medication; and
an antibiotic solution.

8. A soaking operative system for applying a liquid substance comprising:
a hollow member comprising two or more tubes, wherein one or more of the tubes is in a first direction and one or more of the tubes is in a second direction, wherein the one or more tubes in the first direction is connected with the one or more tubes in the second direction at a tube contact point; and
an inlet positioned on one of the tubes, the inlet attachable to a container including the liquid substance.

9. The system of claim 8, wherein the two or more tubes comprise one or more applicator slots and the hollow member is configured to disperse the liquid substance through the one or more applicator slots of the two or more tubes.

10. The system of claim 8, wherein the hollow member is connected to an application needle, wherein the hollow member is configured to be inserted below a patient's skin and the inlet is inserted through the patient skin by an application needle.

11. The system of claim 8, wherein the inlet is connected to a compression connector, the compression connector configured to connect to a syringe, the syringe for providing the liquid substance to the hollow member.

12. The system of claim 8, wherein the two or more tubes of the hollow member are made of an absorbable suture material.

13. The system of claim 8, wherein the two or more tubes comprise a first end and a second end, wherein the first end and the second end of the two or more tubes is sealed.

14. The system of claim 8, wherein the liquid substance comprises at least one or more of:
an anti-inflammatory;
an anesthetic;
an analgesic;
a saline solution;
a chemotherapeutic medication; and
an antibiotic solution.

15. A method of applying a liquid substance with a soaking operative system comprising:
inserting a hollow member below a patient skin, wherein the hollow member comprises two or more tubes, the two or more tubes comprising one or more applicator slots;
delivering the liquid substance to an inlet attached to the hollow member; and
dispersing, by the hollow member, the liquid substance through the one or more applicator slots.

16. The method of claim 15, further comprising:
closing, the patient's skin; and
releasing, by the one or more applicator slots, the liquid substance below the patient skin.

17. The method of claim 15, further comprising:
sealing the hollow member by sealing a first end and a second end of the two or more tubes.

18. The method of claim 15, further comprising:
inserting the inlet through the patient skin by an application needle.

19. The method of claim 15, further comprising:
attaching a compression connector to the inlet; and
sealing, by the compression connector, the inlet.

20. The method of claim 15, further comprising:
dissolving the hollow member, wherein the two or more tubes of the hollow member are made of absorbable suture material.
